(19) United States
(12) Patent Application Publication
(10) Pub. No.: US 2008/0003193 A1
(43) Pub. Date: Jan. 3, 2008

(54) ODOR ELIMINATION AND AIR SANITIZING COMPOSITION

(75) Inventor: Louis Rebrovic, Racine, WI (US)

Correspondence Address: S.C. JOHNSON & SON, INC., 1525 HOWE STREET, RACINE, WI, 53403-2236, US

(73) Assignee: S.C. JOHNSON & SON, INC., Racine, WI (US)

(21) Appl. No.: 11/765,936

(22) Filed: Jun. 20, 2007

Related U.S. Application Data
(60) Provisional application No. 60/817,214, filed on Jun. 28, 2006.

(51) Int. Cl. A61L 9/01; A61L 9/013

(52) U.S. Cl. 424/76.1

(57) ABSTRACT

A deodorizing composition for eliminating airborne and surface malodors is disclosed. The composition comprises triethylene glycol di- and/or mono-glycidyl ether as the malodor counteractant(s). Optionally, the composition may further include triethylene glycol and/or other malodor counteractants. The deodorizing composition eliminates malodors at least by modification reactions between odor-causing molecules and triethylene glycol di- and/or mono-glycidyl ether thereby rendering the odor-causing molecules innocuous.

### CROSS-REFERENCE TO RELATED APPLICATION

This application is a non-provisional U.S. application which is based on and claims priority from provisional Application Ser. No. 60/817,214, filed on Jun. 28, 2006.

### BACKGROUND

1. Technical Field

An improved deodorizing composition is disclosed which effectively removes airborne and surface malodors commonly found in a household. The improved composition includes triethylene glycol diglycidyl ether and/or triethylene glycol monoglycidyl ether as a malodor counteractant.

2. Description of the Related Art

Airborne and surface malodors, when perceived by a person, generally results in detrimental effects to the mental and/or physical wellbeing of that person, particularly when the malodor exists in a household or a business setting where air circulation is insufficient to vent the malodor to an outdoor environment. Common sources of airborne malodors include tobacco smoke, mold and mildew, cooking and/or food odors, pet odors, body odor, etc.

Generally, malodor is the collective olfactory perception of one or more malodorants, which are mainly organic molecules having different structures and functional groups including, but not limited to, amines, acids, alcohols, aldehydes, ketones, phenols, polycyclics, indoles, aromatics, polyaromatics, etc. One particular class of strong malodorants is sulfur-containing compounds, such as mercaptans, sulfides and/or disulfides. In addition to organic molecules, the malodorant can also take inorganic or even elemental forms, such as hydrogen sulfide and chlorine.

Methods and composition for suppressing, eliminating, or counteracting airborne malodors are well known in the art. While a number of physical odor elimination methods, such as by using an air ionizer, has been developed, the most popular and effective odor elimination method is by contacting the malodorant(s) by a deodorizing composition comprising one or more malodor counteractant.

A wide variety of deodorizing compositions are known in the art, the most common of which contain perfumes to mask malodors. Odor masking is the intentional concealment of one odor by the addition of another The masking of odors is typically accomplished by using perfumes or fragrances. However, large quantities of fragrance are needed to ensure that the malodors are no longer noticeable, or are suitably masked. Further, these masking techniques do nothing to remove or modify the source of the malodors.

It has been known to use certain glycols in aerosols or vapor forms to sanitize or disinfect the air in a room by killing airborne bacteria that are often a source of odors. One particular glycol, triethylene glycol (“TEG”), has been found particularly effective for sanitizing air when delivered via an aerosol spray. The commercially successful OUST® air sanitizer products utilize a mixture that contains about 6 wt % TEG. A non-aerosol application of TEG for disinfecting air is also known in the art. TEG has also been used as an air treatment for tobacco smoke.

Triethylene glycol has the following structure:

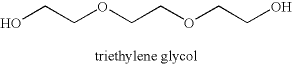

TEG is a colorless, odorless, non-volatile and hygroscopic liquid. It is characterized by two hydroxyl groups along with two ether linkages which contribute to its high water solubility, hygroscopicity and its ability to neutralize airborne odor-causing bacteria in the air. TEG can be prepared commercially by the oxidation of ethylene at high temperatures in the presence of a silver oxide catalyst, followed by hydration of the ethylene oxidate to yield mono-, di-, tri- and tetra-ethylene glycol products. TEG has a low toxicity.

However, TEG is most effective as a malodor counteractant for airborne malodors caused by bacteria. TEG is typically not effective at removing malodors caused by other sources, which could be effectively treated with a modification reaction. Of course, the material used for such a modification reaction would need to be safe for human contact if the material is to be used in air or on surfaces commonly found in a household.

Thus, there is a need for a deodorizing composition that includes a malodor counteractant that can effectively treat a broader range of malodors. Moreover, there is a need for a malodor counteractant that chemically modifies a broad range of malodorants. Finally, there is a need for a deodorizing composition that not only removes malodorants associated with odor-causing microorganisms but also removes a wide spectrum of other malodorants commonly found in a household.

### SUMMARY OF THE DISCLOSURE

In satisfaction of the afore noted needs, a deodorizing composition is disclosed for eliminating airborne and surface malodors commonly found in a household. The composition comprises one or more malodor counteractants each with a molecular structure that includes one or mote glycidyl ether linkages.

In one embodiment, the malodor counteractant is a diglycidyl ether of a glycol. In a refinement, the malodor counteractant is triethylene glycol (TEG) diglycidyl ether TEG diglycidyl ether has the following structure:

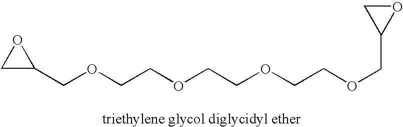

In another embodiment, the malodor counteractant is a monoglycidyl ether of a glycol. In a refinement, the malodor counteractant is TEG monoglycidyl ether. TEG monoglycidyl ether has the following structure:

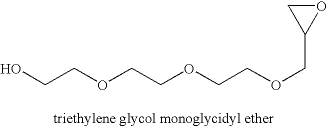

In a preferred embodiment, the disclosed composition comprises a malodor counteractant selected from the group consisting of TEG monoglycidyl ether, TEG diglycidyl ether, and a mixture of both.

In one embodiment, TEG diglycidyl ether and/or TEG monoglycidyl ether may be present in an amount ranging from 0.5 to about 6.0 wt %. Most preferably, the TEG diglycidyl ether and/or TEG monoglycidyl ether comprises about 1 wt % of the formulation.

In addition to the glycidyl ethers, the disclosed composition may optionally include an additional malodor counteractant to improve the odor removing performance thereof. In a preferred embodiment, the additional malodor counteractant is a glycol such as TEG. In a refinement, TEG is present in the disclosed composition in an amount of from about 0.5 to about 6.0 wt %.

Other additional malodor counteractants may also be used in the disclosed composition. In a preferred embodiment, the additional malodor counteractant is be selected from the group consisting of TEG, dipropylene glycol, propylene glycol, and triethanolamine (TEA), and mixtures thereof.

The disclosed composition may take any physical form that is suitable for removing airborne and/or surface malodors. In a preferred embodiment, the disclosed composition is an aqueous solution, emulsion or suspension containing the malodor counteractant. The composition may also be in a form of a solid composite or even exists in a vapor phase.

In a refinement, the disclosed composition is an aqueous composition that includes water as solvent. Preferably, the aqueous composition further includes an organic co-solvent to improve the solubilization of the malodor counteractant and/or other ingredients in the composition.

In a preferred embodiment, the organic co-solvent is a low molecular weight monohydric alcohol. In a further refinement of this concept, the alcohol is selected from a group consisting of ethanol, isopropanol, butanol and propanol. The preferred co-solvent is ethanol due to its low cost and acceptable odor.

The alcohol and water both act as solvents or carriers and the alcohol reduces the drying time of the disclosed liquid formulation. Preferably, the alcohol is a minor component compared to that of water, with the water content ranging from about 75-95 wt % and the alcohol content ranging from about 1 to about 10 wt %, most preferably about 6 wt %.

The disclosed composition may further include one or more surfactants to assist the solubilization of the ingredients included therein and/or to improve the malodor removal performance thereof. The surfactants may comprise one or more nonionic surfactants, a combination of nonionic and ionic surfactants, or more specifically, a combination of nonionic and cationic surfactants. Amphoteric and zwitterionic surfactants may also be used.

The nonionic surfactant preferably comprises a combination of an ether and a hydrogenated oil. In a further refinement, the nonionic surfactants comprise a combination of polyglycol ether and a hydrogenated castor oil. In still a further refinement, polyglycol ether is a polyoxyethylene alkylether.

In a preferred embodiment, the non-ionic surfactant may include (1) a hydrogenated castor oil, that includes glycerol and polyethylene glycol oxystearate, and (2) a polyglycol ether that is an ethoxylation product of C₁₁ to C₁₅ linear secondary alkanols with ethylene oxide.

The ionic surfactant suitable for use in the disclosed composition may comprise a cationic surfactant, such as a quaternary ammonium salt. A benefit of using a quaternary ammonium salt is the anti-bacterial properties of the salt. Other ionic surfactants such as anionic surfactants may also be included in the composition.

In addition to the ingredients discussed above, the disclosed composition may also include other adjuvants including, but not limited to: fragrances, epoxide stabilizers, pH adjusting agents, preservatives, and the like.

In one embodiment, the disclosed composition comprises water, a short chain monohydric alcohol, at least one of TEG diglycidyl ether and TEG monoglycidyl ether or combinations thereof, at least one surfactant, and a fragrance.

In a preferred embodiment, the formulation comprises from about 4 to about 8 wt % ethanol, from about 0.5 to about 1.5 wt % TEG diglycidyl ether and/or TEG monoglycidyl ether, from about 0.5 to about 0.75 wt % fragrance, from about 0.5 to about 1.5 wt % non-ionic surfactant, from about 0.1 to about 1 wt % cationic surfactant and, the remainder, water.

A method of using the disclosed TEG ethers to eliminate airborne and/or surface malodors is disclosed. The method comprises applying a composition containing the TEG ethers to the source of the malodors, and allowing the composition to make contact with the malodors and subsequently eliminate the malodors by chemical modification reactions between the malodorants and the TEG ethers. An exemplary method for preparing the TEG ethers is also disclosed.

Other advantages and features of the disclosed methods and compositions will be described in greater detail below.

### BRIEF DESCRIPTION OF THE DRAWINGS

For a more complete understanding of the disclosed methods and apparatuses, reference should be made to the embodiments illustrated in greater detail in the accompanying drawings, wherein:

FIG. 1 graphically illustrates the ability of the disclosed TEG ethers to reduce the head space concentration of pentanethiol;

FIG. 2 graphically illustrates the ability of the disclosed TEG ethers to reduce the head space concentration of tripropylamine;

It should be understood that the drawings are not necessarily to scale and that the disclosed embodiments are sometimes illustrated diagrammatically and in partial views. In certain instances, details which are not necessary for an understanding of the disclosure may have been omitted. It should be understood, of course, that this disclosure is not limited to the particular embodiments illustrated herein.

### DETAILED DESCRIPTION OF THE PRESENTLY PREFERRED EMBODIMENTS

In a general embodiment, an improved deodorizing composition for eliminating airborne and/or surface malodors is provided. The improved composition includes one or more malodor counteractants that removes airborne and surface malodors. For general purpose of this disclosure, the term “odor removal” refers to the overall reduction of the odor perceived by human sense of smell, and is interchangeable with “odor suppressing”, “odor elimination”, “odor counteracting”, or the like.

Preferably, the malodor counteractant is capable of chemically modifying odor-causing compounds or molecules, i.e. malodorants, associated with the malodor thereby rendering the malodorants innocuous. The malodor counteractants according to this disclosure may include one or more glycidyl ethers of a glycol such as TEG diglycidyl ether and/or TEG monoglycidyl ether, or a mixture of both.

Without being bound to any particular theory, it is believed that the reactive primary epoxy group in TEG di- and/or mono-glycidyl ether reacts with a broad spectrum of malodorants, rendering them innocuous. In particular, malodorants that chemically react with the epoxy group, such as by opening up the three-member ring, may be effectively removed from the air and/or surface.

TEG diglycidyl ether and TEG monoglycidyl ether are non-toxic and safe for low level human contact. Moreover, both ethers have a vapor pressure at room temperature of less than 0.001 mmHg, which allows the malodor counteractant of the composition to make sufficient contact with odor-causing molecules in order to eliminate them by modification reactions.

If a formulation containing one or more glycidyl ethers of TEG is used on a soft surface such as carpeting, upholstery, clothing, bed linens, etc., it is believed that the TEG ethers, in combination with other ingredients of the solution, forms agglomerations within the soft surface. When an odor-causing material or molecule engages these agglomerations, the odor-causing material is dissolved into the agglomeration thereby reducing the partial vapor pressure of the odor-causing material to a level below what is needed to be detected by the human sense of smell. As the TEG ethers or other active ingredient remains in a liquid form, the agglomerations remain as liquid agglomerations for an extended period of time and no dried residue is apparent or visible.

The composition may take any physical form that is suitable for removing airborne and surface malodors. In a preferred embodiment, the disclosed composition is an aqueous solution, emulsion or suspension containing the malodor counteractant. The malodor counteractant may also be incorporated into a solid composite such as by absorbing, adsorbing, or trapping the malodor counteractant in a solid substrate. It is to be understood that the physical presence of the disclosed composition suitable for its application would be apparent to one of ordinary skill in the art and should be considered within the scope of this disclosure.

Six exemplary aqueous composition are listed below. It should be noted that this disclosure is not limited to the particular six formulations and acceptable ranges of the various ingredients are also set forth below.

### EXAMPLE 1

| Wt % | Name/Formula | Function |
|---|---|---|
| 89.735 | de-ionized water | solvent/carrier |
| 6.0 | Ethanol, CH₃CH₂OH | solvent/carrier |
| 1.00 | TEG, triethylene glycol | odor remover |
| | HO(CH₂)₂O(CH₂)₂O(CH₂)₂OH | |
| 1.5 | TEG diglycidyl ether | odor remover |
| | C₁₂H₂₂O₆ | |
| 0.5 | TEG monoglycidyl ether | odor remover |
| | C₉H₁₈O₅ | |
| 0.25 | fragrance oil mixture (TAKASAGO RK- | fragrance |
| | 1428, outdoor) | |
| 0.265 | PROTACHEM ™ CAH-60, hydrogenated | nonionic surfactant |
| | castor oil (glycerol, polyethylene glycol | |
| | oxystearate) | |
| 0.75 | SOFTANOL ™ 70, polyoxyethylene | nonionic surfactant |
| | alkylether | |
| 100.00 | | |

### EXAMPLE 2

| Wt % | Name/Formula | Function |
|---|---|---|
| 89.20 | de-ionized water | solvent/carrier |
| 6.0 | Ethanol, CH₃CH₂OH | solvent/carrier |
| 1.00 | TEG, triethylene glycol | odor remover |
| | HO(CH₂)₂O(CH₂)₂O(CH₂)₂OH | |
| 0.8 | TEG diglycidyl ether | odor remover |
| | C₁₂H₂₂O₆ | |
| 1.2 | TEG monoglycidyl ether | odor remover |
| | C₉H₁₈O₅ | |
| 0.25 | fragrance oil mixture (TAKASAGO RK- | fragrance |
| | 1428, outdoor) | |
| 1.00 | PROTACHEM ™ CAH-60, hydrogenated | nonionic surfactant |
| | castor oil (glycerol, polyethylene glycol | |
| | oxystearate) | |
| 0.55 | TERGITOL ™ 15-s-7, polyglycol ether | nonionic surfactant |
| 100.00 | | |

### EXAMPLE 3

| Wt % | Name/Formula | Function |
|---|---|---|
| 89.40 | de-ionized water | solvent/carrier |
| 6.0 | Ethanol, CH₃CH₂OH | solvent/carrier |
| 1.00 | TEG, triethylene glycol | odor remover |
| | HO(CH₂)₂O(CH₂)₂O(CH₂)₂OH | |
| 1.2 | TEG diglycidyl ether | odor remover |
| | C₁₂H₂₂O₆ | |
| 0.8 | TEG monoglycidyl ether | odor remover |
| | C₉H₁₈O₅ | |
| 0.45 | fragrance oil mixture (TAKASAGO RK- | fragrance |
| | 1428, outdoor) | |
| 0.50 | PROTACHEM ™ CAH-60, hydrogenated | nonionic surfactant |
| | castor oil (glycerol, polyethylene glycol | |
| | oxystearate) | |
| 0.50 | TERGITOL ™ 15-s-7, polyglycol ether | nonionic surfactant |
| 0.15 | Agent 2248-14, quaternary ammonium salt | cationic surfactant |
| 100.00 | | |

### EXAMPLE 4

| Wt % | Name/Formula | Function |
|---|---|---|
| 89.10 | de-ionized water | solvent/carrier |
| 6.0 | Ethanol, CH₃CH₂OH | solvent/carrier |
| 1.00 | TEG, triethylene glycol | odor remover |
| | HO(CH₂)₂O(CH₂)₂O(CH₂)₂OH | |
| 1.0 | TEG diglycidyl ether | odor remover |
| | C₁₂H₂₂O₆ | |
| 1.0 | TEG monoglycidyl ether | odor remover |
| | C₉H₁₈O₅ | |
| 0.40 | fragrance oil mixture (TAKASAGO RK- | Fragrance |
| | 1428, outdoor) | |
| 0.50 | PROTACHEM ™ CAH-60, hydrogenated | nonionic surfactant |
| | castor oil (glycerol, polyethylene glycol | |
| | oxystearate) | |
| 0.50 | TERGITOL ™ 15-s-7, polyglycol ether | nonionic surfactant |
| 0.50 | Agent 2248-14, quaternary ammonium salt | cationic surfactant |
| 100.00 | | |

### EXAMPLE 5

| Wt % | Name/Formula | Function |
|---|---|---|
| 88.60 | de-ionized water | solvent/carrier |
| 6.0 | Ethanol, CH₃CH₂OH | solvent/carrier |
| 1.00 | TEG, triethylene glycol | odor remover |
| | HO(CH₂)₂O(CH₂)₂O(CH₂)₂OH | |
| 1.5 | TEG diglycidyl ether | odor remover |
| | C₁₂H₂₂O₆ | |
| 0.5 | TEG monoglycidyl ether | odor remover |
| | C₉H₁₈O₅ | |
| 0.40 | fragrance oil mixture (TAKASAGO RK- | fragrance |
| | 1428, outdoor) | |
| 0.50 | PROTACHEM ™ CAH-60, hydrogenated | nonionic surfactant |
| | castor oil (glycerol, polyethylene glycol | |
| | oxystearate) | |
| 0.50 | TERGITOL ™ 15-s-7, polyglycol ether | nonionic surfactant |
| 0.40 | Agent 2248-14, quaternary ammonium salt | cationic surfactant |
| 0.60 | Nitrogen | propellant |
| 100.00 | | |

### EXAMPLE 6

| Wt % | Name/Formula | Function |
|---|---|---|
| 79.20 | de-ionized water | solvent/carrier |
| 6.00 | Ethanol, CH₃CH₂OH | solvent/carrier |
| 1.00 | TEG, triethylene glycol | odor remover |
| | HO(CH₂)₂O(CH₂)₂O(CH₂)₂OH | |
| 1.4 | TEG diglycidyl ether | odor remover |
| | C₁₂H₂₂O₆ | |
| 0.6 | TEG monoglycidyl ether | odor remover |
| | C₉H₁₈O₅ | |
| 0.40 | fragrance oil mixture (TAKASAGO RK- | fragrance |
| | 1428, outdoor) | |
| 0.50 | PROTACHEM ™ CAH-60, hydrogenated | nonionic surfactant |
| | castor oil (glycerol, polyethylene glycol | |
| | oxystearate) | |
| 0.50 | TERGITOL ™ 15-s-7, polyglycol ether | nonionic surfactant |
| 0.40 | Agent 2248-14, quaternary ammonium salt | cationic surfactant |
| 10.00 | Hydrocarbon propellant (LPG) | propellant |
| 100.00 | | |

TEG diglycidyl ether and TEG monoglycidyl ether may be used alone or in combination with one another. The two epoxy groups of the TEG diglycidyl ether and the single epoxy group of the TEG monoglycidyl ether react with a broad spectrum of malodorants to render them innocuous. TEG diglycidyl ether is preferred due to its two epoxy groups in comparison to the single epoxy group of TEG monoglycidyl ether. Both are non-toxic and safe for low level human contact.

TEG diglycidyl ether and TEG monoglycidyl ether combined may exceed 5 wt %. In the examples above, the TEG diglycidyl ether and TEG monoglycidyl ether comprise 2.0 wt % of the formulations. However, depending on the particular use or particular odors being treated, the TEG diglycidyl ether and TEG monoglycidyl ether content can vary widely and could range from 0.5 wt % to 6.0 wt % or more Examples 1-6 above, with their combined 2.0 wt %. TEG diglycidyl ether and TEG monoglycidyl ether content, are merely currently preferred embodiments.

As indicated in the above examples, the disclosed composition may optionally include an additional malodor counteractant such as a glycol. The preferred glycol is TEG due to its disinfecting and odor elimination properties. Compositions comprising TEG as the additional malodor counteractant will benefit from at least one, and preferably both, of the following two malodor removal mechanisms: 1) by chemical modification provided by the glycidyl ethers of TEG; and 2) by eliminating odor-causing bacteria and other microorganisms that exist in the air or surface. Depending on the specific malodor, the combination of the two odor elimination mechanisms may create a synergistic effect in removing the malodor.

When included in the aqueous composition, TEG may be present in an amount of from about 0.5 to about 6.0 wt %, more preferably from about 0.5 to about 3 wt %, and most preferably about 1 wt %. It is to be understood that the concentration of TEG is not meant to limit the scope of this disclosure and the inclusion of TEG at other suitable concentrations would be apparent to those of ordinary skill in the art.

It is also noteworthy that TEG is optional and the amount of TEG may be relatively low as compared to an aerosol air sanitizing formulation. Typically, an air sanitizing formulation will have about 6 wt % TEG; in this particular application, i.e., soft surfaces, the amount of TEG can be reduced to less than 5 wt %. In the examples above, the TEG comprises 1.0 wt % of the formulations. However, depending on the particular use or particular odors being treated, the TEG content can vary widely and could range from 0.5 wt % to 6.0 wt % or more. Examples 1-6 above, with their 1.0 wt % TEG content, are merely currently preferred embodiments.

TEG is not the only glycol that can be used to supplement TEG diglycidyl ether and TEG monoglycidyl ether. Dipropylene glycol and propylene glycol are also suitable glycols. Further, the additional malodor counteractant may be any liquid having a vapor pressure at room temperature of less than 0.0035 mmHg such as triethanolamine (TEA).

When the composition is in a form of an aqueous solution, emulsion or suspension, water is included in the composition as a primary solvent. To facilitate the solubilization of the malodor counteractant and other ingredients, the composition may further include an organic co-solvent such as a short chain monohydric alcohol.

The short chain monohydric alcohol is preferably selected from the group consisting of ethanol, propanol, isopropanol, butanol, and isobutanol. In addition to the solubilization benefit, the organic co-solvent also provides for a faster dry time for the applied composition. The most preferred organic co-solvent is ethanol. In a preferred embodiment, the disclosed composition comprises from about 1 to about 10 wt % ethanol.

In addition to the short chain monohydric alcohols, other organic solvents known in the art may also be included in the disclosed composition, such as glycol monoethyl ether or diethylene glycol butyl ether. Preferably, the alcohol or glycol ether co-solvent is present in the composition at a concentration of from about 1 to about 10 wt %. It is to be understood, however, that one of ordinary skill would be able to determine the type and level of the co-solvent that is suitably included in the composition without undue experimentation.

In a further improvement, the disclosed composition may include one or more surfactants. The surfactant may be selected from the group consisting of nonionic surfactant, cationic surfactant, anionic surfactant, amphoteric surfactant, and zwitterionic surfactant. Preferably, the surfactants include at least one nonionic surfactant.

The surfactants used in the disclosed composition may also be known in the art as emulsifiers and, for the purposes of this disclosure, the terms surfactant and emulsifier will be considered to be interchangeable as the common property of surfactants and emulsifiers, i.e., reducing surface tension, is the important function for purposes of this application. Combinations of nonionic surfactants have been found to be effective as well as combinations of nonionic and cationic surfactants.

In a further refinement of this concept, the non-ionic surfactants comprise a combination of a hydrogenated oil and a polyglycol ether. In a further refinement, the ionic surfactant comprises a quaternary ammonium salt. In still a further refinement, the non-ionic surfactants may include (1) a hydrogenated castor oil, that includes glycerol and polyethylene glycol oxystearate, and (2) a polyglycol ether that is a ethoxylation product of C₁₁ to C₁₅ linear secondary alkanols with ethylene oxide.

Examples 1 and 2 utilize only nonionic surfactants in the form of hydrogenated castor oils and polyglycol ethers. The hydrogenated castor oils are sold under the tradename PROTACHEM™ CAH-60 and the INCI/CTFA chemical name “PEG-60 hydrogenated castor oil.” These materials can be obtained from Protameen Chemicals, Inc., 375 Minnisik Road, Totowa, N.J. 07511. The hydrogenated castor oil includes glycerol stearate, and if ethoxylated, includes polyethyelene glycol oxystearate.

The other nonionic surfactant or emulsifier utilized is either TERGITOL™ 15-S-7, which is a polyglycol ether. It is available from Sigma-Aldrich, P.O. Box 14508, St. Louis, Mo. 63718 as well as the Dow Chemical Co., 2030 Dow Center, Midland, Mich. 48674. Other sources of TERGITOL™ will be apparent to those skilled in the art. Another option for a nonionic surfactant is SOFTANOL™ 70, available from Nippon Shokubai of Osaka 541-0043, Japan. Other branched or linear, primary or secondary, polyethoxylated alcohols can be used as nonionic surfactants.

Examples 1 and 2 include only nonionic surfactants. While these examples provide an excellent mechanism for delivering the active ingredients (e.g., TEG diglycidyl ether, TEG monoglycidyl ether and optionally, TEG) and fragrance to odor-causing molecules residing in soft surfaces, it has been surprisingly found that the combination of nonionic and cationic surfactants provides still improved utility. Thus, small amounts of a quaternary ammonium salt in the form of Agent 2248-14 may also be utilized.

The combination of the quaternary ammonium salt with the nonionic surfactants provides improved penetrability and delivery of TEG di- and mono-glycidyl ethers and TEG to the problematic areas within carpeting, upholstery, bedding, drapes, etc. By using a combination of nonionic and cationic surfactants, the disclosed formulations and methods provide a means for delivering TEG di- and mono-glycidyl ethers and TEG to odor-causing molecules buried deep within upholstery or carpeting.

Thus, the malodor counteractant according to this disclosure can be effectively delivered to deeply embedded odor-causing materials such as pet urine or other problematic odors. Further, Agent 2248-14 and other quaternary ammonium salts have anti-microbial properties and therefore add a sanitization function to the disclosed formulations. The total surfactant content preferably ranges from about 0.50 to about 2 wt %.

If utilized, a preferred quaternary ammonium salt (Agent 2248-14) is a mixture of alkyl dimethyl benzyl ammonium chloride and alkyl dimethyl ethyl benzyl ammonium chloride. It is sold by the Stepan Company, 22 Frontage Road, Northfield, Ill. 60093 (www.stepan.com) in a preparation that is 25 wt % alkyl dimethyl benzyl ammonium chloride, 25 wt % alkyl dimethyl ethyl benzyl ammonium chloride, 2-3 wt % ethanol and the remainder water.

A wide variety of cationic surfactants are available and quaternary ammonium salts are not the only possibility. While improving the penetrability of the composition deep into the soft surfaces, the quaternary ammonium salt included in the composition may further improve the odor removal performance of the composition by killing bacteria and other odor-causing microorganisms. While cationic surfactants are utilized in the form of quaternary ammonium salts, other cationic surfactants will be apparent to those skilled in the art without undue experimentation.

In addition to the ingredients discussed above, the disclosed composition may further include additional adjuvants such as fragrances, epoxide stabilizers, pH adjusting agents, preservatives, or the like.

The fragrances utilized can be obtained from Takashago International Corp., a Japanese corporation having an office at 4 Volvo Drive, Rockleigh, N.J. 07647 (http://www.takashago.com). It is to be understood, of course, that one of ordinary skill in the art would be able to determine the type and level of the fragrance that are suitable for use in the disclosed composition and this disclosure is not limited to the particular fragrances utilized herein. A preferred fragrance range is from about 0.25 to about 0.75 wt %.

In another refinement, the applied formulation comprises from about 4 to about 10 wt % low molecular weight monohydric alcohol, from about 0.5 to about 5 wt % glycol, from about 0.5 to about 0.75 wt % fragrance, from about 1 to about 2 wt % nonionic and ionic surfactants and, the remainder, water.

In one preferred embodiment, the formulation comprises from about 4 to about 8 wt % ethanol, from about 0.5 to about 1.5 wt % TEG diglycidyl ether and/or TEG monoglycidyl ether, from about 0.5 to about 0.75 wt % fragrance, from about 0.5 to about 1.5 wt % non-ionic surfactant, from about 0.1 to about 1 wt % cationic surfactant and, the remainder, water.

The disclosed composition may also be in a form of an aerosol as provided in Examples 5 and 6. In such cases, the composition further includes a propellant such as nitrogen propellant, hydrocarbon propellant or other aerosol propellant apparent to one of ordinary skill in the art.

The examples disclosed above are micro emulsions of fragrance, TEG di- and mono-glycidyl ethers, TEG, ethanol and water. When applied, the microemulsion penetrates into the spaces between fibers of a soft surface. Upon evaporation, most of the volatile components (water and ethanol) are removed and a residual agglomeration or droplet of TEG di- and mono-glycidyl ethers, TEG, ethanol, water, fragrance and surfactant serves as an absorbent for odor-causing compounds and molecules. The agglomerations also serve as fragrance extenders.

An exemplary method for preparing the disclosed TEG ethers is discussed in greater detail below. It is to be understood that the preparation of the TEG ethers would be apparent to one of ordinary skill in the art, particularly in view of the method provided herein. Thus, the methods and apparatuses for preparation of the TEG ethers should be considered as within the scope of this disclosure.

### Synthesis of the TEG Ethers

To a 3-necked 250 mL round-bottom flask equipped with mechanical stirrer, reflux condenser topped with a drying tube, and stopper is added 15.02 g (0.100 mol) of TEG and 150 mL of toluene. The resulting mixture is vigorously stirred while 4.96 g of metallic sodium is added in pieces during 1.5 hrs while the stirred mixture is heated to just below reflux. The mixture is then heated under reflux for 2 hrs and then allowed to cool to room temperature (˜25 C.) Thereafter, 32 mL (38 g, 0.41 mol) of epichlorohydrin is added in one portion at room temperature. The reaction mixture was heated under reflux for 2 hrs and then allowed to cool to room temperature. The resulting mixture is then filtered (to remove sodium chloride) and subjected to roto-evaporation to give 26.16 g of a crude product in the form of a viscous yellow oil. The crude product is then subjected to vacuum distillation at ˜1 mm to remove residual volatile materials. The vacuum distilled product may also contain a small amount of TEG monoglycidyl ether (TEGMGE).

Methods and equipments for evaluating the malodorant removal performance of the TEG ethers prepared above ate also provided and discussed in greater detail below.

### Preparation of Air, Saturated with a Malodorant

A 1 L Tedlar Bag with rubber septum (SKC cat # 231-1 or 232-5 septum with single pp fitting with eyelets) is filled to approximately 75% of its total volume with air. The septum is removed and liquid malodorant is added with a micropipette. The rubber septum is then reattached and the bag allowed to sit for 24 hours.

### Head Space Analysis

The removal of the malodors, pentanethiol and tripropylamine, with a test solution of 5 wt % TEG ether in a solvent mixture of 10 wt % EtOH and 90 wt % H2O is measured in the head space of a closed 20 mL rubber septum capped vial. In each case, the concentration of the malodor in the head space is obtained by CC-MS analysis of each sample. The head space concentration of the malodorant over 1 mL of the solvent mixture (10 wt % EtOH/90 wt % H2O) is compared to the head space concentration of the malodorant over 1 mL of the TEG ether test solution. In each case the malodorant is initially at 25% saturation in malodor and the vials are allowed to equilibrate for ˜2 days.

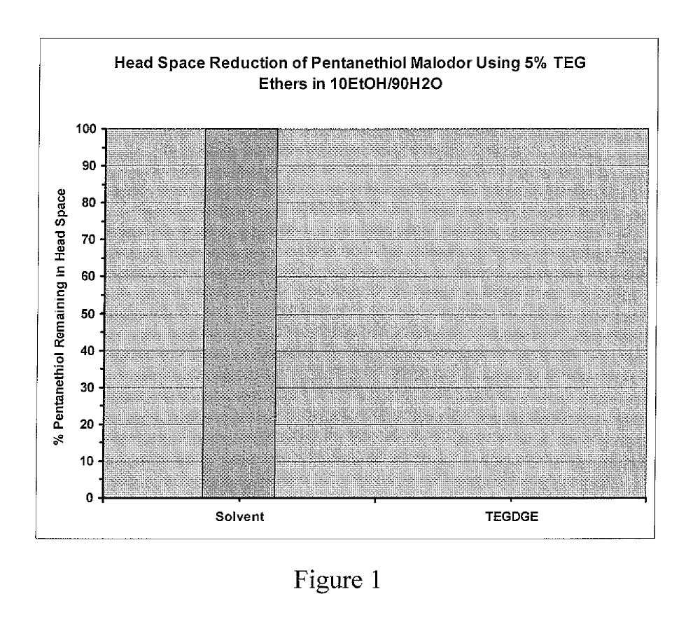

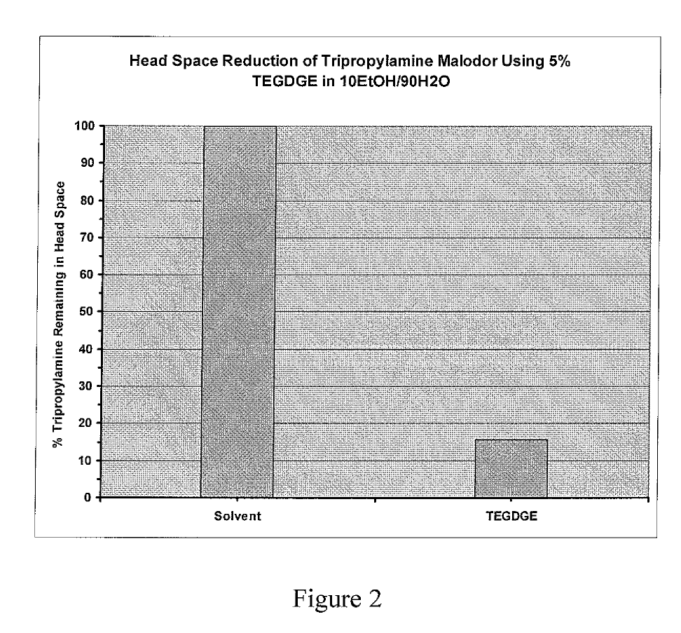

After equilibrium, the head space concentration of the malodorant in each vial is measured by GC-MS. The results are illustrated as bar graphs in FIGS. 1 and 2, wherein the y-axis displays the normalized malodor remaining after equilibration over the solvent or solution.

As clearly indicated in FIGS. 1 and 2, the TEG ether test solution either substantially or completely removes both of the malodorants from the head space of the vials. More specifically, the head space concentration of tripropylamine is reduced by 84%; and the head space concentration of pentanethiol is reduced by 99.99%, indicating excellent odor removal performance of the TEG ethers against at least those two malodorants.

The above discussed evaluation tests are simply purported to demonstrate the efficacy of the TEG ethers in removing malodors and are not meant to limit the scope of the disclosure to the two malodorants discussed above. It is to be understood that one of ordinary skill in the art would be able to recognize malodorants that can be chemically modified by the TEG ethers and the removal the malodorants by using the disclosed composition without undue experimentation.

In summary, the disclosed compositions provide new application of TEG di- and mono-glycidyl ethers as malodor counteractant for airborne and surface malodors, including odors deeply embedded in soft surfaces such as carpeting and upholstery. When the composition is applied, the odor-causing molecules come into contact with and react with the TEG di- and mono-glycidyl ethers and are converted to innocuous materials.

While only certain embodiments have been set forth, alternative embodiments and various modifications will be apparent from the above descriptions to those skilled in the art. These and other alternatives are considered equivalents and within the spirit and scope of this disclosure.

What is claimed:

1. A deodorizing composition for treating malodors, comprising:
at least one of triethylene glycol diglycidyl ether and triethylene glycol monoglycidyl ether.

2. The composition of claim 1 further comprising at least one of a short chain monohydric alcohol and a glycol ether.

3. The composition of claim 2 wherein the alcohol is ethanol and is present in an amount from about 1 to about 10 wt %.

4. The composition of claim 1 further comprising at least one surfactant.

5. The composition of claim 4 wherein the at least one surfactant comprises nonionic and cationic surfactants.

6. The composition of claim 4 wherein at least one surfactant comprises a polyglycol ether and a hydrogenated oil and a quaternary ammonium salt.

7. The composition of claim 6 wherein the hydrogenated oil is a hydrogenated castor oil and the polyglycol ether is a polyoxyethylene alkylether.

8. The composition of claim 1 further comprising an additional malodor counteractant selected from a group consisting of triethylene glycol (TEG), dipropylene glycol, propylene glycol and triethanolamine (TEA).

9. The composition of claim 8 wherein the additional malodor counteractant is triethylene glycol (TEG) and is present from about 0.5 to about 6.0 wt %.

10. The composition of claim 1 wherein the triethylene glycol diglycidyl ether is present in an amount from about 0.5 to about 6.0 wt %.

11. The composition of claim 1 wherein the triethylene glycol monoglycidyl ether is present in an amount from about 0.5 to about 6.0 wt %.

12. The composition of claim 1 wherein a combination of triethylene glycol diglycidyl ether and triethylene glycol monoglycidyl ether is present in an amount from about 0.5 to about 6.0 wt %.

13. A composition for treating household odors, the composition comprising:
at least one of triethylene glycol diglycidyl ether and triethylene glycol monoglycidyl ether;
an additional malodor counteractant selected from the group consisting of triethylene glycol (TEG), dipropylene glycol, propylene glycol and triethanolamine (TEA); and
at least one surfactant.

14. The composition of claim 13 wherein the triethylene glycol diglycidyl ether is present in an amount from about 0.5 to about 6.0 wt %.

15. The composition of claim 13 wherein the triethylene glycol monoglycidyl ether is present in an amount from about 0.5 to about 6.0 wt %.

16. The composition of claim 13 wherein a combination of triethylene glycol diglycidyl ether and triethylene glycol monoglycidyl ether is present in an amount from about 0.5 to about 6.0 wt %.

17. The composition of claim 13 wherein the additional malodor counteractant is triethylene glycol and is present in an amount from about 0.5 to about 6.0 wt %.

18. The composition of claim 13 wherein the at least one surfactant comprises nonionic and cationic surfactants.

19. The composition of claim 18 wherein at least one surfactant comprises a polyglycol ether and a hydrogenated oil and a quaternary ammonium salt.

20. A composition for treating household odors, the composition comprising:
at least one of triethylene glycol diglycidyl ether and triethylene glycol monoglycidyl ether;
an additional malodor counteractant selected from a group consisting of triethylene glycol (TEG), dipropylene glycol, propylene glycol and triethanolamine (TEA);
at least one surfactant;
at least one of a short chain monohydric alcohol and a glycol ether; and
at least one fragrance.
